(19) United States
(12) Patent Application Publication
(10) Pub. No.: US 2013/0023852 A1
(43) Pub. Date: Jan. 24, 2013

(54) Flow Protection Device

(71) Applicant: William Joseph Drasler, Minnetonka, MN (US)

(72) Inventor: William Joseph Drasler, Minnetonka, MN (US)

(21) Appl. No.: 13/549,755

(22) Filed: Jul. 16, 2012

Related U.S. Application Data
(60) Provisional application No. 61/572,858, filed on Jul. 22, 2011.

(51) Int. Cl. A61M 5/165; A61M 25/14

(52) U.S. Cl. 604/508; 604/151

(57) ABSTRACT

A catheter for provides protection to the brachiocephalic and left common carotid arteries against embolic delivery associated with transcatheter aortic valve implantation (TAVI). A catheter having two lumens is introduced into the radial artery and the distal end is delivered to the aorta and an intermediate end is delivered to the brachiocephalic artery. Blood is taken from the aorta and is delivered to the intermediate end via a pump. Fluid flow out of the brachiocephalic artery prevents embolic debris from entering the brachiocephalic artery; streamlines of blood flow into the aorta prevent embolic debris from entering the neighboring left common carotid artery.

### CROSS REFERENCE TO RELATED APPLICATIONS

This patent application makes reference to and incorporates all information found in the provisional patent application No. 61/572,858 entitled Flow Protection Device, filed 22 Jul. 2011 by William J. Drasler.

### FIELD OF THE INVENTION

This invention relates to percutaneous devices and methods used to provide protection to an artery or tubular member of the body, such as a carotid artery, by providing emboli-free fluid flow into the artery to be protected to prevent emboli-laden fluid from entering into the artery or tubular member. The emboli can originate from plaque or calcified lesions which can then be released into the blood stream or fluid stream of the body due to an interventional procedure and can cause ischemic or unwanted results such as a stroke if the embolus travels to a downstream artery that is not protected.

### BACKGROUND OF THE INVENTION

Interventional procedures are often performed in the vascular system or tubular members of the body to correct a stenosis or defect in the tubular member. Such procedures include the placement of stents or performing angioplasty within the coronary or carotid arterial system to correct a stenosis in the arterial lumen. Alternately, interventional procedures are also being performed for transcatheter aortic valve implantation (TAVI). Such procedures can often result in generation of embolic debris from the stenotic aortic valve leaflets or from the diseased aorta. A vast majority of the embolic debris is generated during the 10-20 seconds of the actual deployment of the TAVI device. This embolic debris can travel downstream into the carotid arteries resulting in potential blockage of blood flow to the brain. Such blockages can result in the formation of a transischemic attack (TIA), the creation of a stroke, and possibly can cause death.

Several filter devices have been applied to address this potential embolic problem. Often the filter devices are introduced via a vessel in the patient's arm, such as the radial artery, and through the brachiocephalic artery (BCA) and into the aorta. Alternate filter device designs are introduced through the femoral artery of the leg. A wire frame is often deployed with a mesh material supported by the wire frame. The mesh material in some instances is expected to cover both the BCA and left common carotid artery (LCCA) ostium and deflect embolic material away from the entrance to the BCA or the carotid artery. In other filter devices, the frame and mesh material are deployed within the BCA and LCCA and are designed to filter and collect the embolic debris as blood flow is directed through the mesh material.

Such devices tend to be large in profile and can themselves create emboli from a diseased aorta, from a diseased BCA, or from disease ostii of the BCA or LCCA as they are being deployed. These filter devices can sometimes interfere with the passage of the therapeutic TAVI catheter around the aortic arch. Their placement across the BCA and LCCA for many minutes prior to initiating the TAVI implantation, during the TAVI implantation, and following the interventional procedure can also generate thrombus or thromboemboli due to the small pore size for the mesh material, and the high rate of blood flow through these filters.

Another method of providing protection to the arteries that feed the brain is needed to more efficiently and effectively protect the brain from embolic debris generated during procedures such as the TAVI procedure. The device should have a low profile and should not generate embolic debris as it is being deployed or used.

### SUMMARY

The present invention is a catheter that takes emboli-free blood or fluid from one region of the vascular system or tubular member of the body and delivers it to an artery to be protected or another region of the tubular member of the body to prevent fluid carrying embolic debris from entering into the protected artery. This type of protection can be applied, for example, to protecting the brain from embolic debris by preventing debris-laden blood from entering the BCA or carotid arteries during a TAVI procedure or during the implantation of a carotid stent. The concept can be applied to other regions of the body in order to prevent embolic debris from entering a specific blood vessel or other tubular member of the body.

In one embodiment a catheter is introduced into the right radial artery and advanced through the BCA and into the aorta such that the distal portion of the catheter resides within the aorta. The distal portion has an infusion lumen with a distal opening that allows for blood inflow. A more proximal or dual lumen or intermediate portion of the catheter shaft is positioned such that an intermediate orifice is located within the BCA or near the ostium of the BCA with the aorta. The intermediate orifice communicates with a blood outflow lumen and provides for blood outflow into the artery. The distal opening of the infusion or inflow lumen is located approximately 3-30 cm distal to the intermediate orifice to ensure that blood returning to the blood vessel from the intermediate orifice cannot recirculate to the distal opening of the inflow lumen. Blood is taken from the aorta via the distal opening of the catheter and pulled through the inflow lumen via a pump located outside the body at the proximal end of the catheter. The pump then delivers the blood via the outflow lumen to the intermediate orifice of the catheter; this blood is then directed in a direction to provide blood flow to the BCA and RCA as well as direct flow out of the BCA into the aorta. A filter located in the blood inflow or outflow path ensures that any debris found in the blood is not delivered to the BCA, to either the right or left carotid artery, or to the patient's brain. During the actual 10-20 seconds of the TAVI balloon inflation or implant portion of the procedure, the pump is activated and BCA will thereby receive only filtered blood delivered via the intermediate orifice of the catheter. A fluid such as saline or an oxygenated fluid can alternately or as an auxiliary be supplied from an external supply source and delivered via a pump to the intermediate orifice.

The flow rate of blood that is taken from the aorta and delivered to the BCA is intended to be greater than that normally required to perfuse the brain via the BCA and right carotid artery. The excess blood flow delivered to the BCA will flow from the BCA towards the aorta. This outward flow from the BCA will generate flow streamlines and a boundary layer near the outer curvature of the aorta that will also protect the neighboring left common carotid artery (LCCA) from obtaining embolic debris from a TAVI procedure. Thus any debris generated during a TAVI procedure will not be able to enter either the BCA or the LCCA.

The pump used along with this catheter can be a centrifugal pump, a positive displacement pump, or other pump used in the medical device industry. It is anticipated that the pump will be disposable and operated via a disposable battery.

An alternate embodiment of the present invention has a distal portion of the catheter shaft positioned within the BCA or near the ostium of the BCA and the aorta. A distal opening in the distal portion of the shaft is used to provide blood outflow to the blood vessel. In this embodiment, the more proximal intermediate shaft portion or dual lumen portion has one or more intermediate orifices that provides for blood inflow into the catheter shaft. The proximal shaft portion is positioned within the radial, brachial, axillary, and subclavian arteries. Blood is taken from the radial, brachial, axillary, and subclavian artery via the intermediate orifices for a period of 10-20 seconds and delivered to the BCA and right carotid artery via the distal opening. The blood inflow and outflow is obtained via a pump that is located at the proximal end of the catheter outside of the body. The excess blood flow delivered to the distal catheter opening will flow out of the BCA into the aorta and will protect the LCCA due to creation of a boundary layer of debris-free blood obtained from the distal opening of the present catheter.

### BRIEF DESCRIPTION OF THE DRAWINGS

FIG. 1 is a plan view of the fluid protection catheter having a distal shaft lumen and an intermediate shaft lumen and a pump adjoining the two lumens.

FIG. 2 is a plan view of the fluid protection catheter entering into the radial artery and extending into the brachiocephalic artery and aorta.

FIG. 3 shows the blood flow from the aorta entering the fluid protection catheter and being delivered out of the intermediate orifice creating a streamline the prevents embolic debris from entering the brachiocephalic artery and left common carotid artery.

FIG. 4 shows an alternate embodiment for the fluid protection catheter having blood enter into the intermediate orifice and being pumped via a pump out of the distal opening.

FIG. 5 shows the fluid streamlines generated by the flow pattern of the device of FIG. 4.

### DETAILED DESCRIPTION OF THE INVENTION

The present invention is a low profile interventional catheter which is intended to deliver emboli-free, filtered blood from the aorta to a location within the BCA to protect the brain from emboli that can be generated during a TAVI procedure. The device can also be used in any situation where it is desirable for debris-laden fluid to be prohibited from entering into a vessel or tubular member.

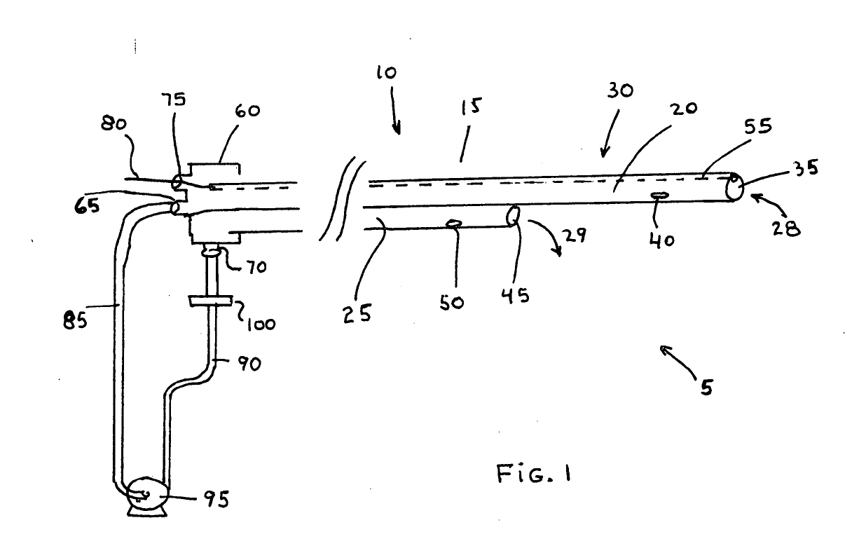

FIG. 1 shows one embodiment of the fluid protection catheter (5) of the present invention. The catheter has an elongated shaft (10) comprised of an intermediate shaft (15) and a distal shaft (30). The intermediate shaft (15) contains at least two lumens, a distal lumen (20) and an intermediate lumen (25). The distal lumen (20) in this embodiment is used for fluid inflow (28) of fluid such as blood or saline or other fluid and the intermediate lumen (25) is used for fluid outflow (29) of fluid such as blood or saline. The distal shaft (30) can have a distal end opening (35) at the end of the distal shaft (30), or it can have one or more distal side openings (40) along the side of the distal shaft (30), or it can have both. The intermediate shaft (15) can have an intermediate end orifice (45) or it can have one or more intermediate side orifices (50), or it can have both. The distal lumen (20) can be used to provide passage over a guidewire (80) or a separate guidewire lumen (55) can be provided along the length of the shaft (10).

The distal end opening (35) is located a distance ranging from 3-30 cm distal to the intermediate orifice (45) such that fluid returning to the blood vessel via the intermediate lumen (25) does not recirculate and enter the distal opening (35) of the distal shaft (30). If the distal opening (35) extends into a large vessel such as the aorta, the distance between the distal opening (35) and the intermediate orifice (45) can be approximately 5-15 cm since the aorta provides a large blood supply; alternately, for a smaller vessel the separation distance should be greater, for example, ranging from 10-30 centimeters.

At the proximal end of the catheter a manifold (60) is located with a distal lumen port (65) that is in fluid communication with the distal lumen (20). Also, the manifold (60) has an intermediate lumen port (70) that is in fluid communication with the intermediate lumen (25). A guidewire port (75) located on the manifold (60) provides passage for a guidewire (80) through the guidewire lumen (55). The guidewire (80) can range from a 0.014 inch to 0.035 inch diameter. The intermediate shaft (15) can range in profile from approximately 4-14 French and the distal shaft (30) can range approximately from 2-8 French and preferably from 2-6 French.

The distal lumen port (65) of this embodiment is attached to a pump inlet tube (85) and the intermediate lumen port (70) can be attached to a pump outflow tube (90). The pump (95) can be a centrifugal pump, a roller pump, piston pump, or other type of pump used to pump blood without generating cellular trauma. A filter (100) can be located in the line with the pump outflow tube (90) or it can be incorporated into the catheter manifold (60) such as at the intermediate lumen port (70), for example. The filter (100) can be formed from a material with a pore size ranging from approximately 10-100 micrometers. The filter (100) can be a fibrous polymer, a sintered metal, sintered plastic, a woven fabric, or other filter (100) structure.

It is understood that the pump (95) can be attached directly to the manifold (60) without the use of pump inflow tube (85) and a pump outflow tube (90); the pump (95) can be connected directly to the distal lumen port (65) and the intermediate lumen port (70). The filter (100) can be incorporated directly into the manifold (60) and can be located in line with the intermediate lumen (25) or the distal lumen (20). The fluid inlet tube (85) can alternately or as an auxiliary be attached to a fluid source such as a saline bag or to an oxygenated fluid source used in medical applications and delivered via the pump (95) to the intermediate end orifice (45).

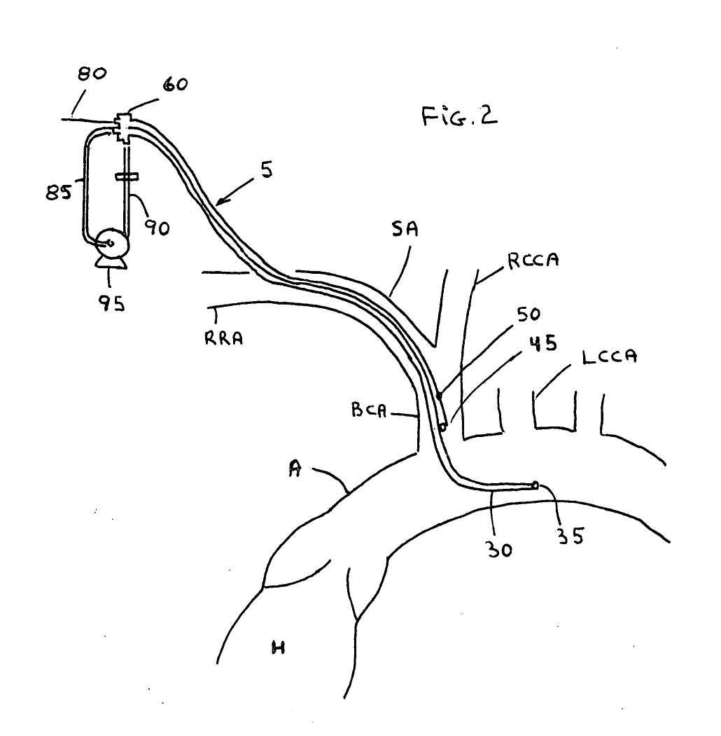

The use of the fluid protection catheter (5) of this embodiment is shown in FIG. 2. The catheter is introduced into the right radial artery (RA) and advanced through the subclavian artery (SA) and through the brachiocephalic artery (BCA) until the distal shaft (30) is located in the aorta (A) with its distal end openings (35) or distal side openings (40) located in the aorta at a distance of at least 5 mm and up to several centimeters. The intermediate shaft (15) does not extend out of the BCA. The intermediate orifice can be located within the BCA near the ostium of the BCA with the aorta or it can be located closer to the junction of the subclavian artery with the right common carotid artery (RCCA).

Upon activation of the pump (95), fluid is drawn into the distal opening and through the distal lumen (20) via the pump inflow tubing (85)to the pump (95). Blood is then delivered through the pump (95) outflow tubing and then through a filter (100) and then back into the intermediate lumen (25) and out of the intermediate orifice into the BCA. The fluid flow delivered out of the intermediate orifice or orifices is enough to provide blood flow to the BCA plus extra to supply the blood flow requirements of the LCCA. In a normal human this blood flow rate could be approximately one liter/minute or 16 cc/sec. For a catheter that is approximately 50 cm to over a 100 cm in overall length, the distal shaft (30) lumen diameter could be less than one mm to effectively draw blood of 3 cp. viscosity into the pump (95) with one atm. driving force. The pump (95) would be activated upon the actual delivery of the TAVI device, which is the cause of the greatest amount of emboli generation. The power to the pump (95) would preferably be provided by a disposable battery; the pump (95) could be powered by standard wall current.

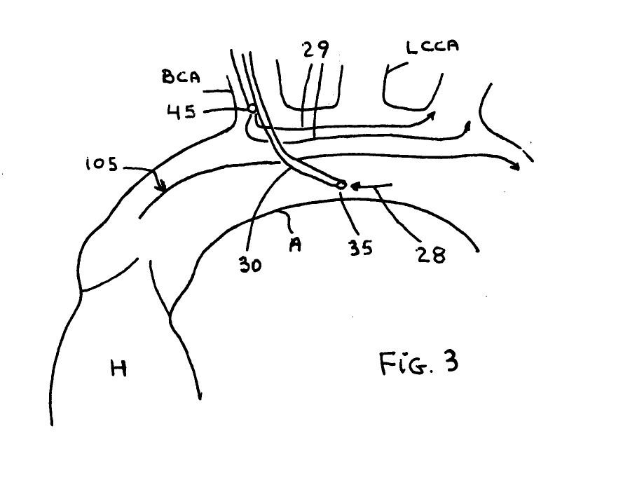

The amount of blood flow inflow into the fluid protection catheter (5) and delivered into the BCA would be greater than that needed to normally supply the BCA. Therefore a portion of the blood delivered out of the intermediate end orifice (45) or intermediate side orifice (50) would flow toward the aorta and into the aorta. This blood flow will cause a boundary layer of debris-free blood to travel along the outer surface of the aorta as shown in FIG. 3. Blood coming from the heart (H) will follow a fluid or blood streamline (105) that is separated from the surface of the aorta. Blood entering the LCCA will therefore be free of embolic debris. The flow from the fluid protection catheter (5) will therefore protect both the RCCA and the LCCA from any debris generated from a TAVI procedure or other procedure that can create emboli upstream of the BCA or SA.

The catheter shaft (10) materials would be similar to standard materials used for diagnostic or therapeutic catheters used in the medical device industry including nylon, polyester, polyethylene, pebax, and other plastics.

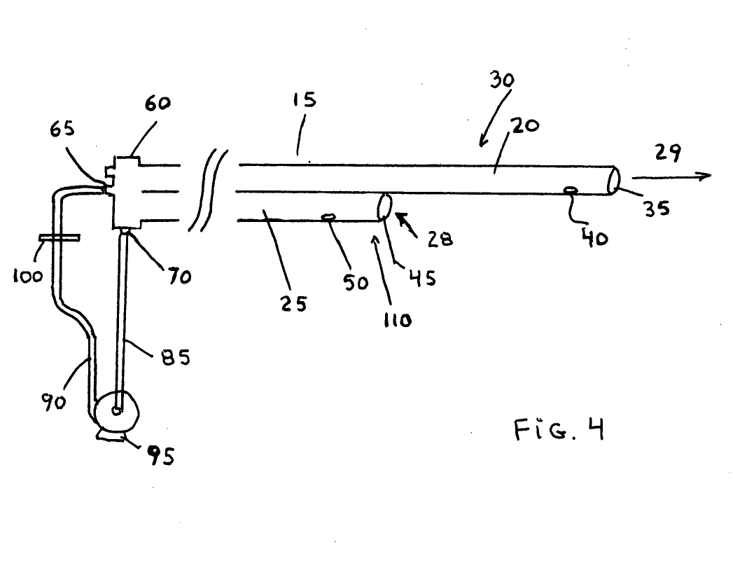

An alternate embodiment for the fluid protection catheter (5) is shown in FIG. 4. In this embodiment the pump (95) is connected opposite to that shown in FIG. 1. The pump outflow tube (90) is connected to the distal lumen port (65) and the pump inflow tube (85) is attached to the intermediate lumen port (70). Blood inflow is directed into the intermediate end orifice (45) or intermediate side orifice (50) or both located at or near the intermediate shaft (15) distal end (110). Blood outflow is returned back to the patient's blood vessel via the distal end opening (35) or distal side opening (40) located at or near the distal shaft (30) distal end. Other components of the device are similar in description to that provided in FIG. 1.

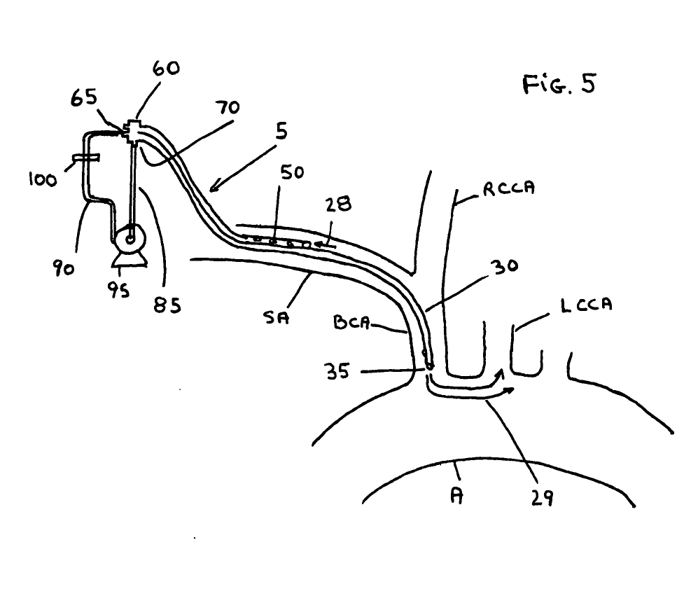

The method of use for this embodiment is shown in FIG. 5. In this embodiment the distal shaft (30) is placed within the BCA or near the ostium of the BCA with the aorta. Blood is drawn into the intermediate lumen (25) via the intermediate end orifices (45) or intermediate side orifices (50) from the subclavian, axillary, brachial and radial arteries as the pump (95) is activated. Blood is pulled into the pump (95) via the intermediate lumen port (70) and through the pump inlet tube (85). Blood is pumped back to the fluid protection catheter (5) via the pump outflow tube (90) to the distal lumen port (65) and into the distal lumen (20) and out of the distal end opening (35) or distal side opening (40). A filter (100) can be placed in the system if desired in the pump outflow tube (90) or near the distal lumen port (65) but the blood found in the subclavian, axillary, brachial, and radial arteries prior to delivery of the TAVI device is expected to be free of embolic debris and filtration may not be required.

Alternate uses of the present invention are anticipated in arteries, veins, and fluid conduits of the body that are required to remain free of embolic debris.

1. A catheter for percutaneous delivery of fluid to a tubular member of the body comprising;
A. a catheter shaft comprising a distal shaft and an intermediate shaft, said distal shaft extending beyond said intermediate shaft portion by a separation distance that does not allow for fluid recirculation between said intermediate and said distal shaft,
B. said distal shaft having a distal shaft lumen that extends from a distal opening located near the distal end of the catheter to a distal lumen port on a manifold located at a proximal end of said catheter,
C. said intermediate shaft having an intermediate lumen that extends from an intermediate orifice near the distal end of said intermediate shaft to an intermediate lumen port on said manifold,
D. said distal lumen port and said intermediate lumen port being connectable to a pump to provide fluid flow through said intermediate lumen in a direction opposed to said distal lumen,
E. a filter in fluid communication with at least one of said intermediate lumen and said distal lumen.

2. The catheter of claim 1 wherein said distal shaft lumen provides for fluid inflow and said intermediate shaft lumen provides for fluid outflow.

3. The catheter of claim 2 wherein said filter is placed in fluid communication with said intermediate shaft lumen.

4. The catheter of claim 1 wherein said distal shaft lumen provides for fluid outflow and said intermediate shaft lumen provides for fluid inflow.

5. The catheter of claim 4 further wherein said filter is placed in fluid communication with said distal shaft lumen.

6. The catheter of claim 1 further comprising a pump that provides fluid flow between said intermediate lumen and said distal lumen.

7. The catheter of claim 1 wherein said separation distance ranges from 3-30 centimeters.

8. The catheter of claim 1 wherein said separation distance ranges from 5-15 centimeters.

9. A catheter for percutaneous delivery of blood to an artery comprising;
A. a catheter shaft having a distal shaft and an intermediate shaft, said distal shaft extending beyond said intermediate shaft by a distance of at least 5 centimeters such that does blood recirculation between said intermediate and said distal shaft does not occur,
B. said distal shaft having a distal shaft lumen that extends from a distal opening located near the distal end of the catheter to a distal lumen port on a manifold located at a proximal end of said catheter,
C. said intermediate shaft having an intermediate lumen that extends from an intermediate orifice near the distal end of said intermediate shaft to an intermediate lumen port on said manifold,
D. said distal lumen port and said intermediate lumen port being connectable to a pump to provide blood flow through said intermediate lumen in a direction opposed to said distal lumen,
E. a filter in fluid communication with said intermediate lumen and said distal lumen.

10. The method of protecting a blood vessel from embolic debris entering the protected blood vessel from an unprotected blood vessel having debris-laden blood, said method comprising;
A. Advancing a catheter comprising a distal shaft having a distal shaft lumen to provide inflow for blood, and an intermediate shaft having an intermediate shaft lumen to provide outflow of blood, said distal shaft extending beyond said intermediate shaft portion by a distance that does not allow for blood recirculation between said intermediate and said distal shaft portions,
B. providing blood inflow into said distal shaft lumen from the debris-laden blood vessel, filtering the embolic debris, and providing blood outflow out of said intermediate shaft lumen to said protected blood vessel.
